(19) United States
(12) Patent Application Publication
(10) Pub. No.: US 2006/0070134 A1
(43) Pub. Date: Mar. 30, 2006

(54) Diagnosis and treatment of fanconi's anemia

(76) Inventors: Tin Tin Su, Boulder, CO (US); Tod Duncan, Boulder, CO (US)

Correspondence Address: SHERIDAN ROSS PC, 1560 BROADWAY, SUITE 1200, DENVER, CO, 80202, US

(21) Appl. No.: 11/203,033

(22) Filed: Aug. 12, 2005

Related U.S. Application Data
(60) Provisional application No. 60/600,898, filed on Aug. 12, 2004.

(51) Int. Cl. A01K 67/027; C12Q 1/68; G01N 33/567; C07H 21/04; C12P 21/06; C12N 9/64; G01N 33/53

(52) U.S. Cl. 800/14; 435/6; 435/69.1; 435/7.2; 435/320.1; 435/325; 435/226; 530/388.26; 536/23.2

(57) ABSTRACT

The novel FLJ11011 gene encoding a human ubiquitin-conjugating (E2) enzyme as well as gene fragments, coding sequences, mRNA variants and encoded proteins are disclosed. Additionally, methods of diagnosing and treating a patient that has or is suspected of having Fanconi's anemia or increased susceptibility to cancer or diminished capability of DNA repair. Additionally, methods of identifying compounds with the potential to treat or ameliorate Fanconi's Anemia are provided.

### CROSS-REFERENCE TO RELATED APPLICATIONS

This application claims the benefit of priority under 35 U.S.C. § 119(e) from U.S. Provisional Application Ser. No. 60/600,898, filed Aug. 12, 2004. The entire disclosure of U.S. Provisional Application Ser. No. 60/600,898 is incorporated herein by reference.

### GOVERNMENT SUPPORT

This invention was made with government support under Grant No. GM66441, awarded by the National Institutes of Health. The government has certain rights in the invention.

### FIELD OF THE INVENTION

This invention is related to diagnostic and prognostic assays as well as treatments for Fanconi's Anemia and cancers and neoplastic disorders related thereto.

### BACKGROUND OF THE INVENTION

Fanconi's anemia (FA) is an autosomal recessive disease associated with chromosomal instability, susceptibility to many forms of cancer including acute myelogenous leukemia (AML) and a plastic anemia. FA occurs equally in males and females and is found in all ethnic groups. Though considered primarily a blood disease, it can affect all systems of the body leading to physical abnormalities including misshapen, missing or extra thumbs, incompletely developed or missing radius, skeletal anomalies of the hips, spine, or ribs, missing or horseshoe kidney and skin discoloration.

Fanconi's anemia has at least 11 complementation groups (A, B, C, D1, D2, E, F, G, I, J, and L), and eight FA genes have been previously cloned. The FA pathway is activated in response to both crosslinked DNA and replication fork stall. Therefore, defects in FA genes are associated with chromosome fragility and decreased cell survival in response to crosslinked DNA. In fact, the definitive test for FA at the present time is a chromosome breakage test in which patient cells are treated with DNA crosslinking agents such as DEB (diepoxybutane) and/or MMC (mitomycin C) and observed for excessive chromosome breakage. Protein ubiquitylation is a recognized signal for protein targeting and protein degradation. Ubiquitin is activated in an ATP-dependent manner by an ubiquitin-activating enzyme known as enzyme-1 (E1), and it is transferred to an ubiquitin-conjugating enzyme (E2) that, with the help of an ubiquitin-protein ligase (E3), specifically attaches ubiquitin to a target protein through the Ε-amino group of a lysine residue. The addition of one ubiquitin residue to proteins in the FA pathway is required to target these proteins to sites of recombinational DNA repair following damage induced by DNA crosslinking agents. The loss of gene function required for this mono-ubiquitinylation is associated with FA and related clinical phenotypes. Alternatively, if the ubiquitin chain is lengthened to at least four sequentially-attached ubiquitins, the target protein is recognized and degraded by the 26S proteasome. Loss of this polyubiquitinylation activity has not been correlated with FA or increased susceptibility to cancer.

The FANCD1 gene, is identical to the breast cancer susceptibility gene, BRCA2, but the function of FANCD1 remains unknown. Similarly, BRCA1 forms a heterodimeric complex that contains an N-terminal zinc ring finger domain that has E3 ubiquitin ligase activity and the loss of the tumor suppressor BRCA1 results in profound chromosomal instability and susceptibility to breast cancer.

Thus, the understanding of the pathogenetic and cellular mechanisms involved in the development of FA remains limited, and therefore, so do therapeutic approaches for preventing and treating the disease.

### SUMMARY OF THE INVENTION

The present invention relates to the discovery of a new gene, FLJ11011, encoding a novel ubiquitin conjugating (E2) enzyme. The invention provides an isolated nucleic acid molecule having a nucleic acid sequence set forth in SEQ ID NO:1, SEQ ID NO:2, SEQ ID NO:3 or SEQ ID NO:4. Additionally, the invention includes an isolated nucleic acid molecule which hybridizes under stringent conditions to a nucleic acid molecule having a sequence set forth in SEQ ID NO:1, SEQ ID NO:2, SEQ ID NO:3 or SEQ ID NO:4. These nucleic acid molecules may be operably linked to a promoter element, comprised in a vector. These vectors may be nucleic acid vectors containing isolated nucleic acids having a nucleic acid sequence set forth in SEQ ID NO:1, SEQ ID NO:2, and SEQ ID NO:3, operatively linked to a heterologous regulatory element and a polyadenylation signal. Alternatively, the nucleic acid constructs may contain a first nucleic acid sequence homologous to a portion of the FLJ11011 gene and a second nucleic acid sequence homologous to a second portion or region of the FLJ11011 gene and a positive selection marker positioned between the first and the second nucleic acid sequences. Preferably, the positive selection marker in these constructs is operatively linked to a promoter and a polyadenylation signal.

Another embodiment of the present invention provides a protein encoded by an isolated nucleic acid molecule having a nucleic acid sequence set forth in SEQ ID NO:1, SEQ ID NO:2, SEQ ID NO:3 or SEQ ID NO:4. These proteins may be peptide fragments composed of at least 20 amino acids that cross-react with an immunoglobulin which specifically binds to a full-length FLJ11011 or CG7220 protein. These proteins may include a protein having the amino acid sequence of SEQ ID NO:5 or a fragment thereof.

Another embodiment of the present invention provides an antibody which binds to a nucleic acid, or to a protein encoded by an isolated nucleic acid molecule, having a nucleic acid sequence set forth in SEQ ID NO:1, SEQ ID NO:2, SEQ ID NO:3 or SEQ ID NO:4. These antibodies may include antibodies that specifically recognize and bind to a fragment of these nucleic acids or proteins. The invention also provides methods of producing antibodies to a ubiquitin-conjugating (E2) enzyme by injecting a compound such as a FLJ11011 gene, a CG7220 gene, a FLJ11011 protein, a CG7220 protein, or fragments of one of these compounds into a host animal, and isolating an antibody that specifically recognizes the compound from the host animal.

In a specific embodiment of the invention, a stem cell is provided that contains a disruption in a FLJ11011 gene. Preferably the stem cell of this embodiment is a human stem cell and the disruption occurs in the FLJ11011 gene.

Another specific embodiment of the invention provides a transgenic mammal comprising a disruption in a FLJ11011 gene. This transgenic mammal lacks production of functional FLJ11011 protein when the disruption in the FLJ11011 gene is homozygous. The invention also includes cells isolated from this transgenic mammal as well as cells that have incorporated a nucleic acid construct containing a recombinase gene operably linked to a functional promoter element.

In a specific embodiment of the invention an isolated cell is provided that contains a FLJ11011 gene capable of expressing proteins and a detectable marker under the control of an inducible promoter.

The present invention also provides methods of identifying agents that are effective in the prevention, treatment or amelioration of symptoms of Fanconi's Anemia. These methods include administering an effective amount of a putative therapeutic agent to a transgenic animal that has a disruption in a FLJ11011 gene, comparing the response of the transgenic animal to a control animal having a functional FLJ11011 gene, wherein a response by the transgenic animal indicative of overcoming or lessening in the symptoms of Fanconi's anemia is indicative of effective treatment of Fanconi's anemia by the agent. In a related embodiment, the invention provides a method of identifying an agent effective for the prevention, treatment or amelioration of symptoms of cancer including administering an effective amount of a putative therapeutic agent to a transgenic animal having a disruption in a FLJ11011 gene, and comparing the response of the transgenic animal to a control animal having a functional FLJ11011 gene, wherein a response by the transgenic animal is indicative of effective treatment or prevention of cancer by the agent.

The present invention also provides methods of identifying an agent effective for the prevention, treatment or amelioration of symptoms of Fanconi's Anemia by contacting an isolated cell having a FLJ11011 gene capable of expressing proteins and a detectable marker under the control of an inducible promoter with a putative therapeutic agent and comparing a cellular phenotype of Fanconi's Anemia of the isolated cell with the phenotype of a control cell lacking a functional FLJ11011 gene, wherein a response by the isolated cell is indicative of an agent that ameliorates symptoms of Fanconi's Anemia. In a related embodiment, the invention provides method of identifying an agent effective for the prevention, treatment or amelioration of symptoms of cancer by contacting an isolated cell having a FLJ11011 gene capable of expressing proteins and a detectable marker under the control of an inducible promoter with a putative therapeutic agent comparing a cellular cancer phenotype of the isolated cell with the phenotype of a control cell lacking a functional FLJ11011 gene, wherein a response by the isolated cell is indicative the prevention or treatment of cancer by the agent.

The present invention also provides methods of testing a mammal for increased susceptibility to cancer by obtaining a tissue sample from the mammal and testing the tissue sample for the presence of a mutation in an FLJ11011 gene. Similarly, the invention provides methods of testing a mammal for increased susceptibility to cancer by obtaining a tissue sample from the mammal and testing the tissue sample for the presence FLJ11011 protein activity in a ubiquitin conjugating assay.

In one specific embodiment of the present invention, a method of diagnosing Fanconi's Anemia (FA) or a predisposition to develop cancer is conducted by detecting in a tissue sample from a patient to be tested the level of expression of FLJ11011, comparing the level of expression of the FLJ11011 detected in the patient sample to a level of expression of FLJ11011 that has been associated with FA and a level of expression of FLJ11011 that has been associated with normal controls and, diagnosing FA in the patient if the expression level of FLJ11011 in the patient sample is statistically more similar to the expression level of the FLJ11011 that has been associated with FA than the expression level of the FLJ11011 that has been associated with the normal controls.

### BRIEF DESCRIPTION OF THE FIGURES OF THE INVENTION

FIG. 1 shows proteins tested in ubiquitinylation reactions transferred from SDS-PAGE to a Ponceau-S stained membrane. Panel A shows a Ponceau-S stained membrane of proteins in ubiquitinylation reactions transferred from an SDS-polyacrylamide gel. Panel B shows that an anti-ubiquitin antibody recognizes poly-ubiquitin conjugates of E3 formed using E1 and Ubc4 and also E1 and CG7220. Panel C is a western blot with an anti-GST antibody showing that the GST-tagged E3 (FANC-L) forms high-molecular weight species in ubiquitinylation reactions using E1 and Ubc4 or CG7220. Molecular weights are shown on the left of each panel in kilodaltons.

FIG. 2 shows proteins from ubiquitinylation reactions separated by SDS-PAGE and transferred to nitrocellulose. Panel A shows an immunoblot using anti-hexahistidine antibodies. Panel B shows an immunoblot using anti-GST antibodies.

FIG. 3 shows Coomassie Blue stained SDS-polyacrylamide gels showing purifications of PHD-domain of Human FA-L (lane A), Hs FLJ11011v2 (lane B), and HsFLJ11011v3 (lane C).

FIG. 4 shows an anti-Ub western blot of ubiqutination reactions containing human E1 and FLJ11011-v2 (lane 1) or FLJ11011-v2 alone (lane 2). The last lane is a positive control for Ub conjugation and western blotting and contains a full reaction with a known E1, E2 (Ubc4) and E3 (FA-L) enzyme.

### DETAILED DESCRIPTION OF THE INVENTION

The present invention relates to the identification and cloning of the Drosophila E2 enzyme, CG7220, that possesses ubiquitin conjugating (E2) activity when used in combination with purified Human E1 and a fragment of the Drosophila homolog of FANC-L (E3). Sequence analysis revealed homologs of CG7220 in the genome of several metazoan species including, C. elegans, Mus musculus, Homo sapiens, Tetraodon nigriviridis and the human homolog, FLJ11011.

Sequence analysis revealed that mRNA encoding FLJ11011 occurs in at least three variant forms having three differently-sized transcripts. When used in ubiquitinylation reactions, a purified protein from one of the variants, FLJ11011v2, shows the ability to accept ubiquitin from an ubiquitin-activating enzyme (E1) in the presence of ATP and is therefore an ubiquitin-conjugating (E2) enzyme. This ubiquitin conjugation of FLJ1011v2 is a covalent modification, but is not dependent upon an E3 (FA-L) protein interaction. Thus, FLJ11011 is a human gene encoding a novel E2 enzyme which is implicated in Fanconi's Anemia (FA) and increased susceptibility to cancer. Sequence analysis from several species showed that FLJ11011 bears approximately 70% identity across the entire predicted amino acid sequence of the proteins.

As used herein, the term “gene” refers to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression including promoters, enhancers and other regulatory sequences.

As used herein, the terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules.

As used herein, “oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine. A “fragment” of a polynucleotide is a polynucleotide comprised of at least 9 contiguous nucleotides, preferably at least 15 contiguous nucleotides and more preferably at least 45 nucleotides, of coding or non-coding sequences.

As used herein, the term “gene targeting” refers to a type of homologous recombination that occurs when a fragment of genomic DNA is introduced into a mammalian cell and that fragment locates and recombines with endogenous homologous sequences.

As used herein, the term “homologous recombination” refers to the exchange of DNA fragments between two DNA molecules or chromatids at the site of homologous nucleotide sequences.

As used herein, the term “homologous” denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology can be determined using a “BLASTN” algorithm. It is to be understood that some functional homologs have less than 40% identity at the amino acid level when compared across very divergent species such as humans and drosophila. It should also be understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

As used herein, the term “target gene” (alternatively referred to as “target gene sequence” or “target DNA sequence” or “target sequence”) refers to any nucleic acid molecule or polynucleotide of any gene to be modified by homologous recombination. The target sequence includes an intact gene, an exon or intron, a regulatory sequence or any region between genes. The target gene comprises a portion of a particular gene or genetic locus in the individual's genomic DNA. As provided herein, the target gene of the present invention is the FLJ11011 gene. A “FLJ11011 gene” refers to an nucleic acid having a sequence that encodes the protein of SEQ ID NO:5 or any sequence that is at least 80% homologous to such a gene sequence

“Disruption” of the FLJ11011 gene occurs when a fragment of genomic DNA locates and recombines with an endogenous homologous sequence. These sequence disruptions or modifications may include insertions, missense, frameshift, deletion, or substitutions, or any combination thereof. Insertions include the insertion of entire genes, which may be of animal, plant, fungal, insect, prokaryotic, or viral origin. Disruption, for example, can alter a FLJ11011 promoter, enhancer, or splice site of the FLJ11011 gene, and can alter the normal gene product by inhibiting its production partially or completely or by enhancing the normal gene product's activity.

The term, “transgenic cell”, refers to a cell containing within its genome the F 11011 gene that has been disrupted, modified, altered completely or partially by the method of gene targeting.

The term “transgenic animal” refers to an animal that contains within its genome a specific gene that has been disrupted by the method of gene targeting. The transgenic animal includes both the heterozygote animal (i.e., one defective allele and one wild-type allele) and the homozygous animal (i.e., two defective alleles). The term “transgenic mouse” or “transgenic mice” refers to a mouse or to mice containing within the genome a specific gene that has been disrupted by the method of gene targeting. The transgenic mouse includes both the heterozygote mouse (i.e., one defective allele and one wild-type allele) and the homozygous mouse (i.e., two defective alleles).

As used herein, the terms “selectable marker” or “positive selection marker” refers to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to those of skill in the art.

As used herein, the term “modulates” refers to the inhibition, reduction, increase or enhancement of the FLJ11011 and/or CG7220 function, expression, activity, or alternatively a phenotype associated with a disruption in the FLJ11011 and/or CG7220 genes.

As used herein, the term “ameliorates” refers to a decreasing, reducing or eliminating of a pathologic condition, disease, disorder, or phenotype, including an abnormality or symptom associated with a disruption in the FLJ11011 and/or CG7220 genes.

The present invention provides nucleic acid molecules having ubiquitin-conjugating (E2) activity, which may include FLJ11011. The nucleic acid molecules of the invention include, but are not limited to, isolated “full-length” nucleic acid molecules which contain, in DNA or RNA form, a complete protein coding sequence, in sense or antisense orientation. The isolated nucleic acid molecules of the invention also include molecules which are not “full-length” but which represent, in DNA or RNA form, a portion of an mRNA molecule, which may constitute protein coding and/or untranslated regions in sense or antisense orientation. Such molecules may be useful as probes for detecting RNA levels, as PCR primers or as antisense inhibitors.

In specific embodiments, the present invention provides isolated nucleic acid molecules having the sequence of SEQ ID NO:1 (GenBank Acc. No. NM001001481), SEQ ID NO:2 (GenBank Acc. No. NM018299), or SEQ ID NO:3 (GenBank Acc. No. NM001001482) which constitute three sequence variants of FLJ11011. A preferred embodiment is the isolated nucleic acid molecule having the sequence of SEQ ID NO:2. The present invention further provides for isolated nucleic acid molecules which hybridize to these nucleic acid molecules under stringent conditions, having lengths of at least 15 nucleotides and preferably at least 50 nucleotides.

In another specific embodiment, the present invention provides for isolated nucleic acid molecules encoding the novel FLJ11011 variants. For example, the present invention provides for isolated nucleic acid molecules including nucleic acid molecules having the sequences set forth as SEQ ID NO:1, SEQ ID NO:2 or as SEQ ID NO:3, or their complementary strands. The present invention further provides for isolated nucleic acid molecules that are between 15 and 500 nucleotides in length, preferably between 50 and 1000 nucleotides in length, and more preferably between 1000 and 10,000 nucleotides in length, which hybridize to a molecule having the sequences set forth in SEQ ID NO:1, SEQ ID NO:2 or SEQ ID NO:3 or their complementary strands under stringent conditions. A related embodiment is an isolated nucleic acid molecule having an amino acid sequence having at least 40% homology to the nucleic acid sequence set forth in SEQ ID NO:1, SEQ ID NO:2 or SEQ ID NO:3 and more preferably, 70% homology, and more preferably, 80% homology, and more preferably, 85% homology, and more preferably, 90% homology, and more preferably, 95% homology, and most preferably, 100% homology.

Another embodiment of the present invention is a nucleic acid molecule which hybridizes under stringent conditions to a nucleic acid molecule having a sequence as set forth in SEQ ID NO:1, SEQ ID NO:2 or SEQ ID NO:3. A related embodiment is an isolated nucleic acid molecule which hybridizes under stringent conditions to an amino acid sequence having at least 40% homology to SEQ ID NO: 1, SEQ ID NO: 2 or SEQ ID NO: 3 and more preferably, 70% homology, and more preferably, 80% homology, and more preferably, 85% homology, and more preferably, 90% homology, and more preferably, 95% homology, and most preferably, 100% homology.

The present invention further provides for isolated nucleic acid molecules which encode a protein having an amino acid sequence as set forth as SEQ ID NO:5, their complementary strands, and nucleic acid molecules which hybridize under stringent conditions to their sense or antisense strands.

In a specific embodiment, the present invention provides an isolated nucleic acid molecule for the Drosophila E2 gene, CG7220 (GenBank Acc. No. NM136760), or an isolated nucleic acid molecule having an amino acid sequence having at least 40% homology to SEQ ID NO:4, and more preferably, 70% homology, and more preferably, 80% homology, and more preferably, 85% homology, and more preferably, 90% homology, and more preferably, 95% homology, and most preferably, 100% homology to SEQ ID NO:4.

Another embodiment of the present invention is a nucleic acid molecule which hybridizes under stringent conditions to a nucleic acid molecule having a sequence as set forth in SEQ ID NO:4 or an isolated nucleic acid molecule which hybridizes under stringent conditions to an amino acid sequence having at least 40% homology to SEQ ID NO:4 and more preferably, 70% homology, and more preferably, 80% homology, and more preferably, 85% homology, and more preferably, 90% homology, and more preferably, 95% homology, and most preferably, 100% homology. The present invention further provides for isolated nucleic acid molecules which hybridize to these nucleic acid molecules under stringent conditions, having lengths of at least 15 nucleotides and preferably at least 50 nucleotides.

In another specific embodiment, the present invention provides for isolated nucleic acid molecules encoding variants of the Drosophila E2 gene, or their complementary strands and isolated nucleic acid molecules which encode the protein products of the nucleic acid sequence set forth as SEQ ID NO:4, its complementary strand, and nucleic acid molecules which hybridize under stringent conditions to its sense or antisense strands.

Any of the isolated nucleic acid molecules of the present invention may be linked to a heterologous nucleic acid. For example a FLJ11011 nucleic acid molecule may be engineered such that it is in an “expressible form” meaning a form in which a FLJ11011 nucleic acid molecule is linked to one or more elements necessary or desirable for transcription and/or translation and/or purification. For example, the isolated FLJ11011 nucleic acid molecule may be operatively linked to a suitable promoter element in an expression cassette which may further comprise a transcription initiation and termination site, nucleic acid encoding a nuclear localization sequence, a ribosome binding site, a polyadenylation site, and/or an mRNA stabilizing sequence. Examples of suitable promoter elements, include, but are not limited to, the cytomegalovirus immediate early promoter, the Rous sarcoma virus long terminal repeat promoter, the human elongation factor-1 a promoter, the human ubiquitin c promoter, and the like. It may be desirable, in certain embodiments of the invention, to use a promoter whose regulation can be controlled. Examples of such promoters include the murine mammary tumor virus promoter (inducible with dexamethasone), commercially-available steroid- or tetracycline-responsive promoters, or ecdysone-inducible promoters. It may further be desirable, in certain embodiments of the invention, to use FLJ11011 -specific promoters. Other suitable constitutive, regulatable, or cell- or tissue-specific promoter systems are known to those of ordinary skill in the art.

A nucleic acid molecule of the invention, whether or not it is to be expressed as a protein, may be inserted into a suitable vector for duplication purposes. Suitable vectors include, but are not limited to, plasmids, cosmids, phages, phagemids, artificial chromosomes, replicons, and various virus-based vector systems known in the art.

Where it is desired to express a FLJ11011 or CG7220 protein or peptide, suitable expression vectors include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN, lentiviruses, for example human immunodeficiency virus (“HIV”), feline leukemia virus (“FIV”) or equine infectious anemia virus (“EIAV”)-based vectors, adenoviruses, adeno-associated viruses, herpes simplex viruses, for example vectors based on HSV-1, baculoviruses, SV40, Epstein-Barr viruses, alphaviruses, vaccinia viruses or any other class of viruses that can efficiently transduce human tumor cells and that can accommodate the nucleic acid sequences required for therapeutic efficacy.

Examples of non-virus-based delivery systems that may be used according to the invention include, but are not limited to, so-called “naked nucleic acids,” meaning nucleic acids encapsulated in liposomes, nucleic acid/lipid complexes and nucleic acid/protein complexes.

The ubiquitin-conjugating (E2) enzymes of the present invention may also be produced using isolated nucleic acids molecules contained in plasmids, such as pCEP4, pMAMneo and pcDNA3.1. Vectors useful in expressing ubiquitin-conjugating (E2) enzymes of the present invention in bacterial systems include, but are not limited to, the GST vector and the chitin binding domain vector (TYB-12).

In a preferred embodiment, a ubiquitin-conjugating (E2) enzyme vector comprises the FLJ11011 coding sequence of SEQ ID NO:2 operatively linked to a heterologous regulatory element and a polyadenylation signal, both of which are active in mammalian cells. The resulting expression cassette may be contained within a plasmid or a virus-based vector.

The present invention provides for ubiquitin-conjugating proteins. These include, but are not limited to, the protein products encoded by the CG7220 and FLJ11011 genes. The invention also encompasses peptide fragments of these proteins comprising at least 20 amino acids which cross-react with an immunoglobulin which specifically binds to the corresponding full-length protein.

In a specific embodiment, the present invention provides for proteins encoded by nucleic acids having a nucleic acid sequence set forth in SEQ ID NO:2 or SEQ ID NO:4, and more preferably for a protein having an amino acid sequence as set forth in SEQ ID NO:5. The invention further encompasses peptide fragments of such proteins comprising at least 20 amino acids that cross-react with an immunoglobulin which specifically binds to a full-length FLJ11011 or CG7220 protein.

The ubiquitin-conjugating proteins and peptides of the invention may be prepared by standard techniques, including recombinant DNA-related techniques and chemical synthesis, or by collection from natural sources. For recombinant DNA expression, suitable expression vectors are set forth above. Expression systems which may be used to produce ubiquitin conjugating proteins include prokaryotic and eukaryotic expression systems, including eukaryotic cells, bacteria, fungi (e.g. yeast), insects, and the like. Depending on the expression system used, a nucleic acid may be introduced by any standard technique, including transfection, transduction, electroporation, bioballistics, or microinjection.

The invention also provides a targeting construct and methods of producing the targeting construct that, when introduced into stem cells, produces a homologous recombinant. In one embodiment, the targeting construct of the present invention comprises first and second polynucleotide sequences that are homologous to the FLJ11011 gene. The targeting construct also comprises a polynucleotide sequence that encodes a selectable marker that is preferably positioned between the two different homologous polynucleotide sequences in the construct. The targeting construct may also comprise other regulatory elements that may enhance homologous recombination.

Targeting constructs of the present invention may be produced using standard methods known in the art. (See, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional means, wherein sequences may be synthesized, isolated from natural sources, manipulated, cloned, ligated, subjected to in vitro mutagenesis, primer repair, or the like. At various stages, the joined sequences may be cloned and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present invention typically comprises a first sequence homologous to a portion or region of the FLJ11011 gene and a second sequence homologous to a second portion or region of the FLJ11011 gene. The targeting construct further comprises a positive selection marker, which is preferably positioned in between the first and the second DNA sequences that are homologous to a portion or region of the target DNA sequence. The positive selection marker may be operatively linked to a promoter and a polyadenylation signal. Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Similarly, a targeting construct of the present invention may also include a sequence homologous to a portion or region of the CG7220 gene and a second sequence homologous to a second portion or region of the CG7220 gene. The targeting construct may further comprise a positive selection marker, which may be operatively linked to a promoter and a polyadenylation signal, other regulatory sequences and a sequence coding for a screening marker.

Although the size of the homologous sequence is not critical (ranging from as few as 50 base pairs to as many as 100 kb) preferably each fragment is greater than about 1 kb in length, more preferably between about 1 and about 10 kb, and even more preferably between about 1 and about 5 kb. One of skill in the art will recognize that although larger fragments may increase the number of homologous recombination events in embryonic stem cells, larger fragments will also be more difficult to clone.

Once an appropriate targeting construct has been prepared, the targeting construct may be introduced into an appropriate host cell using any method known in the art. Various techniques may be employed in the present invention, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyornithine, or the like (See, e.g., U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (See, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In a preferred embodiment of the present invention, the targeting construct is introduced into host cells by electroporation. In this process, electrical impulses of high field strength reversibly permeabilize biomembranes allowing the introduction of the construct. The pores created during electroporation permit the uptake of macromolecules such as DNA. (See, e.g., Potter, H., et al., 1984, Proc. Nat'l. Acad. Sci. U.S.A. 81:7161-7165).

Any cell type capable of homologous recombination may be used in the practice of the present invention. Examples of such target cells include cells derived from vertebrates including mammals such as humans, bovine species, ovine species, murine species, simian species, and other eucaryotic organisms such as filamentous fungi, and higher multicellular organisms such as plants. Preferably, the cells of the present invention are derived from murine species.

Preferred cell types include embryonic stem cells, which are typically obtained from pre-implantation embryos cultured in vitro. (See, e.g., Evans, M. J., et al., 1981, Nature 292:154-156; Bradley, M. O., et al., 1984, Nature 309:255-258; Gossler et al., 1986, Proc. Natl Acad. Sci. USA 83:9065-9069; and Robertson, et al., 1986, Nature 322:445-448). The embryonic stem cells are cultured and prepared for introduction of the targeting construct using methods well known to the skilled artisan. (See, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorin et al., 1992, Transgenic Res. 1:101; and Veis et al., 1993, Cell 75:229). The embryonic stem cells that will be inserted with the targeting construct are derived from an embryo or blastocyst of the same species as the developing embryo into which they are to be introduced. Embryonic stem cells are typically selected for their ability to integrate into the inner cell mass and contribute to the germ line of an individual when introduced into the mammal in an embryo at the blastocyst stage of development. Thus, any embryonic stem cell line having this capability is suitable for use in the practice of the present invention.

The present invention may also be used to knockout genes in cell types, such as stem cells. By way of example, stem cells may be bone marrow progenitor and precursor cells. These cells, comprising a disruption or knockout of a gene, may be particularly useful in the study of FLJ11011 gene function in ubiquitinylation or DNA repair pathways. Stem cells may be derived from any vertebrate species, such as mouse, rat, dog, cat, pig, rabbit, human, non-human primates and the like. Preferably the stem cells are derived from mouse.

After the targeting construct has been introduced into cells, the cells in which successful gene targeting has occurred are identified. Insertion of the targeting construct into the targeted gene is typically detected by identifying cells for expression of the marker gene. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

If a regulated positive selection method is used in identifying homologous recombination events, the targeting construct is designed so that the expression of the selectable marker gene is regulated in a manner such that expression is inhibited following random integration but is permitted (de-repressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (See, e.g., Mansour et al., Nature 336:348-352: (1988); Capecchi, Science 244:1288-1292, (1989); Capecchi, Trends in Genet. 5:70-76 (1989)).

Successful recombination may be identified by analyzing the DNA of the selected cells to confirm homologous recombination. Various techniques known in the art, such as PCR and/or Southern analysis may be used to confirm homologous recombination events. Homologous recombination may also be used to disrupt genes in stem cells, and other cell types, which are not totipotent embryonic stem cells.

In cells that are not totipotent it may be desirable to knock out both copies of the target using methods that are known in the art. For example, cells comprising homologous recombination at a target locus that have been selected for expression of a positive selection marker (e.g., Neo^{r}) and screened for non-random integration, can be further selected for multiple copies of the selectable marker gene by exposure to elevated levels of the selective agent (e.g., G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target.

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected embryonic stem cells can be allowed to aggregate with dissociated mouse embryo cells to form the aggregation chimera. A chimeric embryo can then be implanted into a suitable pseudopregnant female foster animal and the embryo brought to term. Chimeric progeny harboring the homologously-recombined DNA in their germ cells can be used to breed animals in which all cells of the animal contain the homologously recombined DNA. In one embodiment, chimeric progeny mice are used to generate a mouse with a heterozygous disruption in the FLJ11011 gene. Heterozygous transgenic mice can then be mated. It is well known in the art that typically one-quarter of the offspring of such matings will have a homozygous disruption of at least one of these genes.

Heterozygous and homozygous transgenic mice can be compared to normal, wild type mice to determine whether disruption of the FLJ11011 gene causes phenotypic changes resembling FA in humans, especially pathological changes such as increased susceptibility to cancer and/or excessive chromosome breakage. For example, heterozygous and homozygous mice may be evaluated for phenotypic changes by physical examination, necropsy, histology, clinical chemistry, complete blood count, body weight, organ weights, and cytological evaluation of bone marrow.

The present invention further contemplates conditional transgenic or knockout animals, such as those produced using recombination methods. Bacteriophage P1 Cre recombinase and flp recombinase from yeast plasmids are two examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present invention. Such recombinases include the Int recombinase of bacteriophage λ (with or without Xis) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec.Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al.,(Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxp site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. For example, when the constructs described herein are used to disrupt a FLJ11011 gene, a fusion transcript can be produced when insertion of the positive selection marker occurs downstream (3′) of the translation initiation site of the FLJ11011 gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In one embodiment, purified recombinase enzyme is provided to the cell by direct microinjection. In another embodiment, recombinase is expressed from a co-transfected construct or vector in which the recombinase gene is operably linked to a functional promoter.

An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al. ,Proc. Natl. Acad. Sci. USA, 91:9302-6 (1994)).

Additional control elements that can be used include promoters requiring specific transcription factors such as viral promoters. Vectors incorporating such promoters only express recombinase activity in cells that express the necessary transcription factors.

The cell- and animal-based systems described herein can be utilized as models for diseases. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify compounds or compositions that are capable of ameliorating symptoms of FA, decreasing susceptibility to cancer and/or increasing susceptibility of cancer cells to DNA crosslinking by anti-cancer agents such as mitomycin C, cisplatin or diepoxybutane. Thus, the animal- and cell-based models of the present invention may be used to identify drugs, pharmaceuticals, therapies and interventions that may be effective in treating or preventing FA and/or cancer.

Cell-based systems may be used to identify compounds that may act to ameliorate FA symptoms. For example, such cell systems may be exposed to a compound suspected of exhibiting an ability to ameliorate symptoms of FA, at a sufficient concentration and for a time sufficient to elicit such an amelioration of FA symptoms in the exposed cells. After exposure, the cells are examined to determine whether one or more of the cellular phenotypes of FA has been altered to resemble a more normal or more wild type, non-disease phenotype.

In addition, animal-based FA model systems, such as those described herein, may be used to identify compounds capable of ameliorating FA symptoms. Such animal models may be used as test substrates for the identification of drugs, pharmaceuticals, therapies, and interventions that may be effective in treating FA or other phenotypic characteristics of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with FA. Exposure may involve treating mother animals during gestation of the model animals described herein, thereby exposing embryos or fetuses to the compound or agent that may prevent or ameliorate the FA or symptoms thereof. Neonatal, juvenile, and adult animals can also be exposed.

More particularly, using the animal models of the invention, specifically, transgenic mice, methods of identifying agents, on the basis of their ability to affect at least one phenotype associated with a disruption in the FLJ11011 gene are provided. In one embodiment, the present invention provides a method of identifying agents having an effect on FLJ11011 expression or function. The method includes measuring a physiological response of the animal, for example, to the agent, and comparing the physiological response of such animal to a control animal, wherein the physiological response of the animal comprising a disruption in the FLJ11011 gene as compared to the control animal indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time) and cellular assays (e.g., immunohistochemical assays of cell surface markers, or the ability of cells to aggregate or proliferate) can be used to assess a physiological response. The transgenic animals and cells of the present invention may be utilized as models for diseases, disorders, or conditions associated with phenotypes relating to a disruption in the FLJ11011 gene.

FANCD2 is one of the proteins in the D complementation group of the FA pathway. FANCD2 is the endpoint of the FA pathway and is not part of the FA nuclear complex nor required for its assembly or stability. FANCD2 exists in two isoforms, FANCD2-S and FANCD2-L. Ubiquitinylation transforms the protein short form (FAND2-S) to the protein long form (FANCD2-L) and occurs in response to the FA complex. Defects in proteins associated with the FA pathway result in a failure to make the ubiquitinylated form, FANCD2-L, and failure to make FANCD2-L correlates with errors in DNA repair and cell cycle abnormalities associated with FA. Thus, the function of the FLJ11011 gene product can be assessed by the ubiquitinylation of FANCD2-S to FANCD2-L. Therefore, one embodiment of the present invention provides a method of assessing the function of a FLJ11011 gene and/or gene product in a host by assaying for the ubiquitinylation of the FANCD2 protein. Methods of assaying and monitoring this ubiquitinylation reaction are described in U.S. patent application Publication No. 20030188326, which is incorporated herein in its entirety. In this embodiment, the ubiquitinylation of a FANCD2 protein of a host cell or mammal is assayed by these known methods, wherein the absence of the formation of the ubiquitinylated FANCD2-L protein is indicative of a disruption in the FLJ11011 gene, whereas successful ubiquitinylation of the FANCD2 enzyme is indicative of a functional FLJ11011 gene.

The present invention provides for the use of FLJ11011 and CG7220 gene sequences to produce FLJ11011 and/or CG7220 gene products. Such gene products may include proteins that represent functionally equivalent gene products. Such an equivalent gene product may contain deletions, additions or substitutions of amino acid residues within the amino acid sequence encoded by the gene sequences described herein, but which result in a silent change, thereby producing a functionally equivalent FLJ11011 and/or CG7220 gene product. Amino acid substitutions may be made on the basis of similarities in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar activity as the endogenous gene products encoded by the FLJ11011 or CG7220 sequences. Alternatively, when utilized as part of an assay, “functionally equivalent” may refer to peptides capable of interacting with other cellular or extracellular molecules in a manner substantially similar to the way in which the corresponding portion of the endogenous gene product would.

Additional protein products useful according to the methods of the invention are peptides derived from, or based on, the FLJ11011 and/or CG7220 genes produced by recombinant or synthetic means (derived peptides). These gene products may be produced by recombinant DNA technology using techniques well known in the art. Thus, methods for preparing the gene polypeptides and peptides of the invention by expressing nucleic acid encoding gene sequences are described herein. Methods that are well known to those skilled in the art can be used to construct expression vectors containing FLJ11011 protein coding sequences and appropriate transcriptional/translational control signals. These methods include, for example, in vitro recombinant DNA techniques, synthetic techniques and in vivo recombination/genetic recombination (See, e.g., Sambrook, et al., 1989, supra, and Ausubel, et al., 1989, supra). Alternatively, RNA capable of encoding FLJ11011 protein sequences may be chemically synthesized using, for example, automated synthesizers (See, e.g. Oligonucleotide Synthesis: A Practical Approach, Gait, M. J. ed., IRL Press, Oxford (1984)).

A variety of host-expression vector systems may be utilized to express the gene coding sequences of the invention. Such host-expression systems represent vehicles by which the coding sequences of interest may be produced and subsequently purified, but also represent cells that may, when transformed or transfected with the appropriate nucleotide coding sequences, exhibit the protein of the invention in situ. These include, but are not limited to, microorganisms such as bacteria (e.g., E. coli, B. subtilis) transformed with recombinant bacteriophage DNA, plasmid DNA or cosmid DNA expression vectors containing FLJ11011 protein coding sequences; yeast (e.g. Saccharomyces, Pichia) transformed with recombinant yeast expression vectors containing the gene protein coding sequences; insect cell systems infected with recombinant virus expression vectors (e.g., baculovirus) containing the gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing gene protein coding sequences; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors that direct the expression of high levels of fusion protein products that are readily purified may be desirable. Such vectors include, but are not limited to, the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned protein can be released from the GST moiety.

In an insect system, Autographa califomica nuclear polyhedrosis virus (AcNPV) may be used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (See, e.g., Smith, et al., J. Virol. 46: 584-93 (1983); U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing FLJ11011 protein in infected hosts. (e.g., see Logan et al., Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter, et al., Methods in Enzymol., 153:516-44 (1987)).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, and WI38.

For long-term, high-yield production of recombinant proteins, stable expression is preferred. For example, cell lines that stably express the FLJ11011 protein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells that stably integrate the plasmid into their chromosomes and grow, to form foci, which in turn can be cloned and expanded into cell lines. This method may advantageously be used to engineer cell lines that express the FLJ11011 protein. Such engineered cell lines may be particularly useful in screening and evaluation of compounds that affect the endogenous activity of the gene protein.

In a preferred embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the FLJ11011 protein may be labeled, either directly or indirectly, to facilitate detection of a complex formed between the FLJ11011 protein and a test substance. Any of a variety of suitable labeling systems may be used including but not limited to radioisotopes such as I¹²⁵; enzyme labeling systems that generate a detectable calorimetric signal or light when exposed to substrate; and fluorescent labels. Where recombinant DNA technology is used to produce the FLJ11011 protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include, but are not limited to, polyclonal, monoclonal, chimeric, single chain, Fab fragments and fragments produced by a Fab expression library.

Described herein are methods for the production of antibodies capable of specifically recognizing one or more epitopes. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of the FLJ11011 and/or CG7220 genes in a biological sample, or, alternatively, as a method for the inhibition of abnormal or normal activity of one or more of these genes or the proteins encoded by these genes. Additionally, the homology of the Drosophila and human ubiquitin-conjugating genes (FLJ11011 and CG7220) is at least 70% identical and therefore, antibody probes will cross-react across species of mouse and human. Thus, such antibodies may be utilized as part of disease treatment methods and/or may be used as part of diagnostic techniques whereby patients may be tested for abnormal levels of FLJ11011 and/or CG7220 proteins, or for the presence of abnormal forms of such proteins.

For the production of antibodies, various host animals may be immunized by injection with a gene, its expression product or a portion thereof. Such host animals may include, but are not limited to, rabbits, mice, rats, goats and chickens. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as the FLJ11011 gene product or an antigenic functional derivative thereof. For the production of polyclonal antibodies, host animals such as those described above, may be immunized by injection with a gene product supplemented with adjuvants as described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

The present invention may be employed in a process for screening for agents such as agonists, i.e. agents that bind to and activate FLJ11011 and/or CG7220 polypeptides, or antagonists, i.e. agents that inhibit the activity or interaction of FLJ11011 and/or CG7220 polypeptides with its ligand. Thus, polypeptides of the invention may also be used to assess the binding of small molecule substrates and ligands in, for example, cells, cell-free preparations, chemical libraries, and natural product mixtures as known in the art. Any methods routinely used to identify and screen for agents that can modulate gene expression may be used in accordance with the present invention.

The present invention provides methods for identifying and screening for agents that modulate expression or function of FLJ11011 and/or CG7220. More particularly, cells that contain and express FLJ11011 and/or CG7220 gene sequences may be used to screen for therapeutic agents. Such cells may include non-recombinant monocyte cell lines, such as U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as HeLa cells and COS cells, e.g., COS-7 (ATCC# CRL-1651).

Further, such cells may include recombinant, transgenic cell lines. For example, the transgenic mice of the invention may be used to generate cell lines, containing one or more cell types involved in FA, that can be used as cell culture models for that cancer susceptibility disorder. While cells, tissues, and primary cultures derived from the transgenic animals of the invention may be utilized, the generation of continuous cell lines is preferred. For examples of techniques that may be used to derive a continuous cell line from the transgenic animals, see Small, et al., Mol. Cell Biol., 5:642-48 (1985).

FLJ11011 and/or CG7220 gene sequences may be introduced into, and overexpressed in, the genome of the cell of interest. In order to overexpress the gene sequence of interest, the coding portion of the gene sequence may be ligated to a regulatory sequence that is capable of driving gene expression in the cell type of interest. Such regulatory regions will be well known to those of skill in the art, and may be utilized in the absence of undue experimentation.

FLJ11011 and/or CG7220 gene sequences may also be disrupted or underexpressed. Cells having disruptions in, or underexpressing, FLJ11011 and/or CG7220 gene sequences may be used, for example, to screen for agents capable of affecting alternative pathways that compensate for any loss of function attributable to the disruption or underexpression. The FLJ11011 or CG7220 genes may be inhibited using biomolecules that disrupt the expression or function of these genes including, but are not limited to, FLJ11011 or CG7220 antisense nucleic acids (antisense FLJ11011 or CG7220 gene RNAs, oligonucleotides, modified oligonucleotides, or RNAi), inhibitors of FLJ11011 or CG7220 gene transcription, mRNA processing, mRNA transport, protein translation, or protein modification.

Patients displaying or at risk for developing FA may be treated by gene therapy. One or more copies of normal FLJ11011 and/or CG7220 genes, or a portion of these genes that directs the production of a normal protein with wild-type gene function, may be inserted into the patient's cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal FLJ11011 and/or CG7220 gene sequences into human cells.

In vitro systems may be designed to identify compounds capable of binding the FLJ11011 and/or CG7220 gene products. Such compounds may include, but are not limited to, peptides made of D-and/or L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; See, e.g., Songyang, et al., Cell, 72:767-78 (1993)), antibodies, and small organic or inorganic molecules. Compounds identified may be useful, for example, in modulating the activity of a FLJ11011 protein, preferably mutant FLJ11011 proteins; elaborating the biological function of the FLJ11011 protein; or screening for compounds that disrupt normal FLJ11011 gene interactions or themselves disrupt such interactions.

The principle of the assays used to identify compounds that bind to the FLJ11011 and/or CG7220 proteins involves preparing a reaction mixture of the respective protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring either or both proteins or the test substance onto a solid phase and detecting target protein/test substance complexes anchored on the solid phase at the end of the reaction. In one embodiment of such a method, the FLJ11011 and/or CG7220 proteins may be anchored onto a solid surface, and the test compound, which is not anchored, may be labeled, either directly or indirectly.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for FLJ11011 or CG7220 gene products or the test compound to anchor any complexes formed in solution, and a labeled antibody specific for the other component of the possible complex to detect anchored complexes.

Compounds that are shown to bind to a particular gene product through one of the methods described above can be further tested for their ability to elicit a biochemical response from interaction with the respective FLJ11011 and/or CG7220 proteins. Agonists, antagonists and/or inhibitors of the expression product can be identified utilizing biochemical assays of FA pathway proteins well known in the art.

Additionally, the use of any of the known biological assays that test the function of the FA pathway to ubiquitinylate proteins or to repair DNA damage caused by DNA cross linking agents can be used to assess the function of the FLJ11011 and CG7220 genes and proteins. Further, host cells deficient in one or both of these activities can be used to assay for the successful incorporation and expression of the FLJ11011 and CG7220 genes and proteins. In this way, the biochemical activity associated with functional FA pathway genes and proteins can be used as a screen for compounds that interfere with, enhance the activity of, or restore the function to, cells having incorporated FLJ11011 or CG7220 genes.

Other agents that may be used as therapeutics include the FLJ11011 and/or CG7220 genes, their expression products and functional fragments thereof. Additionally, agents that reduce or inhibit mutant FLJ11011 gene activity may be used to ameliorate FA symptoms. Such agents include antisense, ribozyme, and triple helix molecules. Techniques for the production and use of such molecules are well known to those of skill in the art.

Anti-sense RNA and DNA molecules ( including antisense FLJ11011 gene RNAs, oligonucleotides, modified oligonucleotides, RNAi) act to directly block the translation of mRNA by hybridizing to targeted mRNA and preventing protein translation. In the case of RNAi, they also act to promote RNA degradation. With respect to antisense DNA, oligodeoxyribonucleotides derived from the translation initiation site, e.g., between the −10 and +100 regions of the FLJ11011 gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to at lest one of FLJ11011 and CG7220 mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding FLJ11011 and/or CG7220 proteins.

Specific ribozyme cleavage sites within any potential RNA target are initially identified by scanning the molecule of interest for ribozyme cleavage sites that include the following sequences, GUA, GWU and GUC. Once identified, short RNA sequences of between 15 and 20 ribonucleotides corresponding to the region of the gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant FLJ11011 and/or CG7220 gene alleles. In order to ensure that substantially normal levels of gene activity are maintained, nucleic acid molecules that encode and express gene polypeptides exhibiting normal activity may be introduced into cells that do not contain sequences susceptible to whatever antisense, ribozyme, or triple helix treatments are being utilized. Alternatively, it may be preferable to co-administer normal gene proteins into the cell or tissue in order to maintain the requisite level of cellular or tissue gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include, but are not limited to, the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for the FLJ11011 protein, and in particular, mutant gene protein, and interfere with its activity may be used to inhibit FLJ11011 or mutant FLJ11011 gene function. Such antibodies may be generated against the proteins themselves or against peptides corresponding to portions of the proteins using standard techniques known in the art and as also described herein. Such antibodies include, but are not limited to, polyclonal, monoclonal, Fab fragments, single chain antibodies and chimeric antibodies.

In instances where the FLJ11011 gene protein is intracellular and whole antibodies are used, internalizing antibodies may be preferred. However, lipofectin liposomes may be used to deliver the antibody or a fragment of the Fab region that binds to the FLJ11011 gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the FLJ11011 gene protein may be used. Such peptides may be synthesized chemically or produced via recombinant DNA technology using methods well known in the art (See, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular FLJ11011 gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

RNA and/or DNA sequences encoding FLJ11011 and/or CG7220 proteins may be directly administered to a patient exhibiting FA symptoms, at a concentration sufficient to produce a level of FLJ11011 proteins such that FA symptoms are ameliorated. Patients may be treated by gene therapy. One or more copies of a normal FLJ11011 gene, or a portion of the gene that directs the production of a normal FLJ11011 protein with FLJ11011 gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal FLJ11011 gene sequences into human cells.

Cells, preferably autologous cells, containing normal gene-expressing gene sequences may then be introduced or reintroduced into the patient at positions that allow for the amelioration of FA or FA symptoms.

The identified compounds that inhibit target mutant gene expression, synthesis and/or activity can be administered to a patient at therapeutically effective doses to treat or ameliorate FA. A therapeutically effective dose refers to that amount of the compound sufficient to result in amelioration of symptoms of FA.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically-acceptable carriers or excipients. Thus, the compounds and their physiologically-acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the treatments may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulation agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

Oral ingestion is possibly the easiest method of taking any medication. Such a route of administration, is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, very little or any of a wide variety of biologically active proteinaceous material, if simply taken orally, would survive its passage through the stomach to be taken up by the body in the small intestine. The result is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration.

In another preferred embodiment, the pharmaceutical compositions of the present invention are formulated to deliver a target enzyme involved in ubiquitinylation such as the proteins encoded by SEQ ID NO:2 or SEQ ID NO:4, to a patient with FA. The proteins may be administered by any acceptable route of administration. Preferably, the protein(s) are administered intravenously or intramuscularly in a therapeutically-effective dosage in single or divided doses. The pharmaceutical formulations used to administer the proteins to the mammal in need of such treatment may include agents that increase the absorption of proteins or inhibit the digestion or hydrolysis of these proteins.

A variety of methods may be employed to diagnose disease conditions associated with the FLJ11011 gene. Specifically, reagents may be used, for example, for the detection of the presence of FLJ11011 mutations, alternations, deletions or duplications, or the detection of either over- or under-expression of mRNA, or aberrant or absent FLJ11011 protein activity in tissues or serum. These changes in the FLJ11011 gene, mRNAs or proteins may be indicative of a patient's susceptibility to FA or cancer, the diagnosis of FA or cancer or evaluative of the progress of a patient's FA or cancer.

For example, one embodiment of the present invention relates to a method to diagnose Fanconi's Anemia (FA) or a predisposition to develop cancer. The method includes detecting in a tissue sample from a patient to be tested the level of expression of FLJ11011, comparing the level of expression of the FLJ11011 detected in the patient sample to a level of expression of FLJ11011 that has been associated with FA and a level of expression of FLJ11011 that has been associated with normal controls and diagnosing FA in the patient if the expression level of FLJ11011 in the patient sample is statistically more similar to the expression level of the FLJ11011 that has been associated with FA than the expression level of the FLJ11011 that has been associated with the normal controls.

In one aspect of this embodiment, the method includes detecting the expression of at least one gene chosen from a gene containing, or expressing a transcript containing, a nucleic acid sequence that may include SEQ ID NOs:1-4.

Various techniques can be used to detect the expression of the gene or genes including, but not limited to, measuring amounts of transcripts of the gene in the patient peripheral blood cells, detecting hybridization of at least a portion of the gene or a transcript thereof to a nucleic acid molecule comprising a portion of the gene or a transcript thereof in a nucleic acid array, or using quantitative polymerase chain reaction (q-PCR). In one embodiment, expression of the gene is detected by detecting the production of a protein encoded by the gene.

According to the diagnostic and prognostic methods of the present invention, alteration of the wild-type FLJ11011 gene locus is detected. In addition, the method can be performed by detecting the wild-type FLJ11011 gene locus and confirming the lack of a predisposition or a FA phenotype. “Alteration of a wild-type gene” encompasses all forms of mutations including deletions, insertions and point mutations in the coding and noncoding regions. Deletions may be of the entire gene or only a portion of the gene. Point mutations may result in stop codons, frameshift mutations or amino acid substitutions. Somatic mutations are those that occur only in certain tissues, but are not inherited in the germline. Germline mutations can be found in any bodily tissues and are inherited. If only a single allele is mutated, a heterozygous state is indicated. However, if both alleles are mutated, then an expectation of FA is indicated. The finding of gene mutations thus provides both diagnostic and prognostic information. A gene allele that is not deleted (e.g., that is found on the sister chromosome to a chromosome carrying the FLJ11011 gene deletion) can be screened for other mutations, such as insertions, small deletions, and point mutations. Mutations leading to non-functional gene products may also be linked to FA and/or cancer susceptibility. Point mutational events may occur in regulatory regions, such as in the promoter of the gene, leading to loss or diminution of expression of the mRNA. Point mutations may also abolish proper RNA processing, leading to loss of expression of the FLJ11011 gene products, or a decrease in mRNA stability or translation efficiency. The FLJ11011 protein levels may also be reduced by promoter hypermethylation and this may be subsequently associated with pre-disposition to FA and associated disorders. Methods of measuring DNA methylation of genes are well known in the art (see, for example, U.S. Pat. Nos. 6,200,756; 6,331,393; 6,251,594 which are incorporated herein by reference in their entirety). Therefore, one embodiment of the present invention is a method of assessing the methylation state of the FLJ11011 or CG7220 genes. In a preferred embodiment, the invention provides microarrays of tissue biopsy samples from patients being treated with one or more chemotherapy compounds for the determination of the methylation state of the FLJ11011 gene as a measurement of the degree of a tumor's resistance to one or more chemotherapy compounds.

The methods described herein may be performed, for example, by utilizing pre-packaged diagnostic kits comprising at least one specific gene nucleic acid or anti-gene antibody reagent described herein, which may be conveniently used, e.g., in clinical settings, to diagnose patients exhibiting early FA symptoms or at risk for developing FA. Any cell type or tissue, preferably platelets, neutrophils or lymphocytes, in which the gene is expressed may be utilized in the diagnostic testing.

DNA or RNA from the cell type or tissue to be analyzed may easily be isolated using procedures that are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992).

Gene nucleotide sequences, either RNA or DNA, may, for example, be used in hybridization or amplification assays of biological samples to detect FA-related gene structures and expression. Such assays may include, but are not limited to, Southern or Northern analyses, restriction fragment length polymorphism assays, single stranded conformational polymorphism analyses, in situ hybridization assays, and polymerase chain reaction analyses. Such analyses may reveal both quantitative aspects of the expression pattern of the gene, and qualitative aspects of the gene expression and/or gene composition. That is, such aspects may include, for example, point mutations, insertions, deletions, chromosomal rearrangements, and/or activation or inactivation of gene expression.

Preferred diagnostic methods for the detection of gene-specific nucleic acid molecules may involve for example, contacting and incubating nucleic acids, derived from the cell type or tissue being analyzed, with one or more labeled nucleic acid reagents under conditions favorable for the specific annealing of these reagents to their complementary sequences within the nucleic acid molecule of interest. Preferably, the lengths of these nucleic acid reagents are at least 9 to 30 nucleotides. After incubation, all non-annealed nucleic acids are removed from the nucleic acid:fingerprint molecule hybrid. The presence of nucleic acids from the fingerprint tissue that have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art.

Alternative diagnostic methods for the detection of gene-specific nucleic acid molecules may involve their amplification, e.g., by PCR (the experimental embodiment set forth in Mullis U.S. Pat. No. 4,683,202 (1987)), ligase chain reaction (Barany, Proc. Natl. Acad. Sci. USA, 88:189-93 (1991)), self sustained sequence replication (Guatelli, et al., Proc. Natl. Acad. Sci.

USA, 87:1874-78 (1990)), transcriptional amplification system (Kwoh, et al., Proc. Natl. Acad. Sci. USA, 86:1173-77 (1989)), Q-β Replicase (Lizardi et al., Bio/Technology, 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment of such a detection scheme, a cDNA molecule is obtained from an RNA molecule of interest (e.g., by reverse transcription of the RNA molecule into cDNA). Cell types or tissues from which such RNA may be isolated include any tissue in which wild type FLJ11011 gene is known to be expressed, including, but not limited to, platelets, neutrophils and lymphocytes. A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method may be chosen from among the gene nucleic acid reagents described herein. The preferred lengths of such nucleic acid reagents are at least 15-30 nucleotides. For detection of the amplified product, the nucleic acid amplification may be performed using radioactively or non-radioactively labeled nucleotides. Preferably, fluorophore-quench conjugated primers are used in assessing mRNA expression measured by the well-known methods of quantitative polymerase chain reaction (q-PCR) or quantitative reverse transcriptase-polymerase chain reaction (qRT-PCR). Alternatively, enough amplified product may be made such that the product may be visualized by standard ethidium bromide staining or by utilizing any other suitable nucleic acid staining method.

Antibodies directed against wild type or mutant gene peptides may also be used as FA diagnostics and prognostics. Such diagnostic methods may be used to detect abnormalities in the level of gene protein expression, or abnormalities in the structure and/or tissue, cellular, or subcellular location of FLJ11011 gene protein. Structural differences may include, for example, differences in the size, electronegativity, or antigenicity of the mutant FLJ11011 gene protein relative to the normal FLJ11011 gene protein.

Protein from the tissue or cell type to be analyzed may easily be detected or isolated using techniques that are well known to those of skill in the art, including, but not limited to, western blot analysis. The protein detection and isolation methods employed herein may also be such as those described in Harlow and Lane, for example, (Antibodies: A Laboratory Manual, Cold Spring Harbor Laboratory Press, Cold Spring Harbor, N.Y. (1988)).

Preferred diagnostic methods for the detection of wild type or mutant gene peptide molecules may involve, for example, immunoassays wherein fingerprint gene peptides are detected by their interaction with an anti-fingerprint gene-specific peptide antibody.

For example, antibodies, or fragments of antibodies useful in the present invention may be used to quantitatively or qualitatively detect the presence of wild type or mutant gene peptides. This can be accomplished, for example, by immunofluorescence techniques employing a fluorescently labeled antibody (see below) coupled with fluorescence microscopy, flow cytometric, or fluorimetric detection. Such techniques are especially preferred if the fingerprint gene peptides are expressed on the cell surface.

The antibodies (or fragments thereof) useful in the present invention may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the fingerprint gene peptides, but also their distribution in the examined tissue. Using the present invention, those of ordinary skill will readily perceive that any of a wide variety of histological methods (such as staining procedures) can be modified in order to achieve such in situ detection.

Immunoassays for wild type, mutant, or expanded fingerprint gene peptides typically comprise incubating a biological sample, such as a biological fluid, a tissue extract, freshly harvested cells, or cells that have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled gene-specific antibody. The solid phase support may then be washed with the buffer a second time to remove unbound antibody. The amount of bound label on solid support may then be detected by conventional means.

The terms “solid phase support or carrier” are intended to encompass any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet or test strip. Preferred supports include polystyrene beads. Those skilled in the art will know many other suitable carriers for binding antibody or antigen, or will be able to ascertain the same by use of routine experimentation.

The binding activity of anti-wild type or -mutant fingerprint gene peptide antibody may be determined according to well known methods. Those skilled in the art will be able to determine operative and optimal assay conditions for each determination by employing routine experimentation.

One of the ways in which the gene peptide-specific antibody can be detectably labeled is by linking the same to an enzyme and using it in an enzyme immunoassay (EIA) (Voller, Ric Clin Lab, 8:289-98 (1978) “The Enzyme Linked Immunosorbent Assay (ELISA),” Diagnostic Horizons 2:1-7, 1978, Microbiological Associates Quarterly Publication, Walkersville, Md.; Voller, et al., J. Clin. Pathol., 31:507-20 (1978); Butler, Meth. Enzymol., 73:482-523 (1981); Maggio (ed.), Enzyme Immunoassay, CRC Press, Boca Raton, Fla. (1980); Ishikawa, et al., (eds.) Enzyme Immunoassay, Igaku-Shoin, Tokyo (1981)). The enzyme that is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type, mutant, or expanded peptides through the use of a radioimmunoassay (RIA) (See, e.g., Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediamine-tetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### EXAMPLES

### Example 1

### Identification and Testing of a Ubiquitin Conjugating Enzyme from Drosophila

Drosophila CG7220 was identified as a putative ubiquitin-conjugating enzyme (E2). Recombinant purified E1 (ubiquitin activating), Ubc4 (ubiquitin conjugating) and E3 (ubiquitin ligating) enzymes were purified and used to test the ability of CG7220 to act as an E2 enzyme (i.e. to substitute for Ubc4). Briefly, 20 μl reactions were prepared containing recombinant E1 (50 to 500 nM; Calbiochem), Ubc4 protein (0.5 to 5 μM; Boston Biochem) or isolated Drosophila CG7220 GST fusion protein, Ubiquitin (5 μM; Boston Biochem), and ATP (2 mM) in buffer. The reactions were incubated at 23° C. for 90 min and stopped by the addition of an equal volume of 2×SDS sample buffer. The protein-ubiquitin conjugates were analyzed on SDS PAGE on a 12% gel and Western blotting with an anti-GST or anti-Ubiquitin antibody.

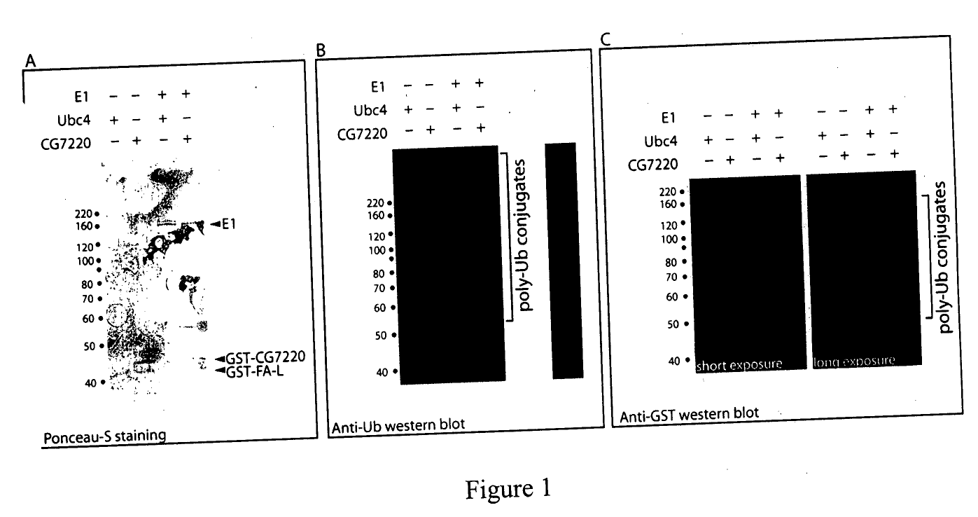

Referring to FIG. 1, Panel A shows a Ponceau-S stained membrane of proteins in ubiquitinylation reactions transferred from an SDS-polyacrylamide gel. Panel B shows that an anti-ubiquitin antibody recognizes poly-ubiquitin conjugates of E3 formed using E1 and Ubc4 and also E1 and CG7220. Panel C is a western blot with an anti-GST antibody showing that the GST-tagged E3 (FANC-L) forms high-molecular weight species in ubiquitinylation reactions using E1 and Ubc4 or CG7220. Molecular weights are shown on the left of each panel in kilodaltons.

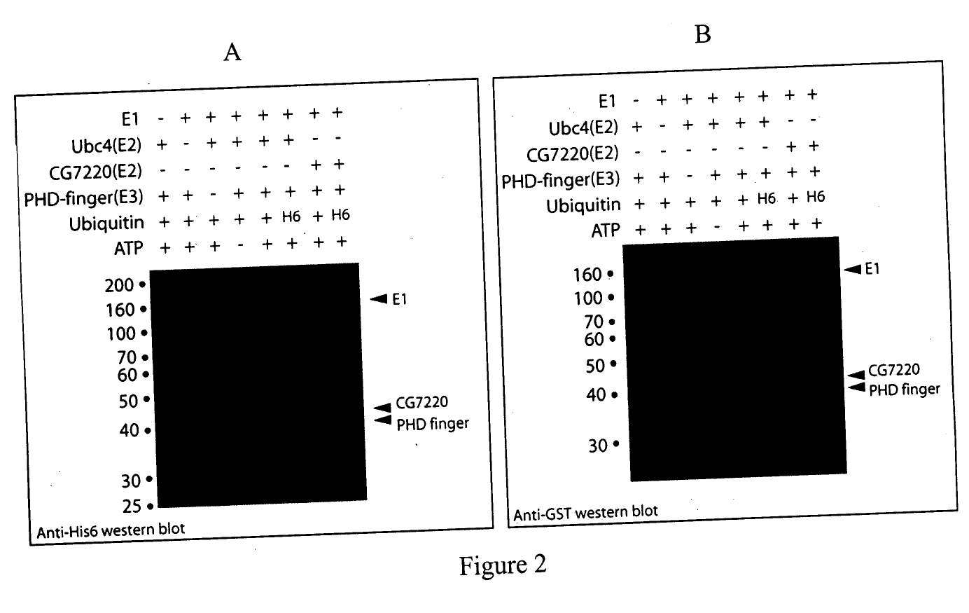

Referring to FIG. 2, Panel A shows proteins in ubiquitinylation reactions separated by SDS-PAGE, transferred to nitrocellulose membrane and immunoblotted with anti-hexahistidine antibodies. The antibody specifically recognizes hexahistidine tagged ubiquitin which is conjugated to either CG7220 or E3 enzymes. It is also shown that this reaction is dependent on an active E1, E3 and ATP. Panel B shows identical reactions immunoblotted with an anti-GST antibody. E1, CG7220 and E3 (PHD-finger) are all GST tagged. High molecular weight GST-tagged species are formed in reactions containing E1, Ubc4 or CG7220 and E3. Molecular weights are shown to the left of each panel in kilodaltons. The migration position through SDS-PAGE of the E1, CG7220 and E3 are indicated to the right of the panel. Ubc4 is not shown on these immunoblots.

### Example 2

### Identification of Human Genomic Sequence with Homology to Drosophila E2

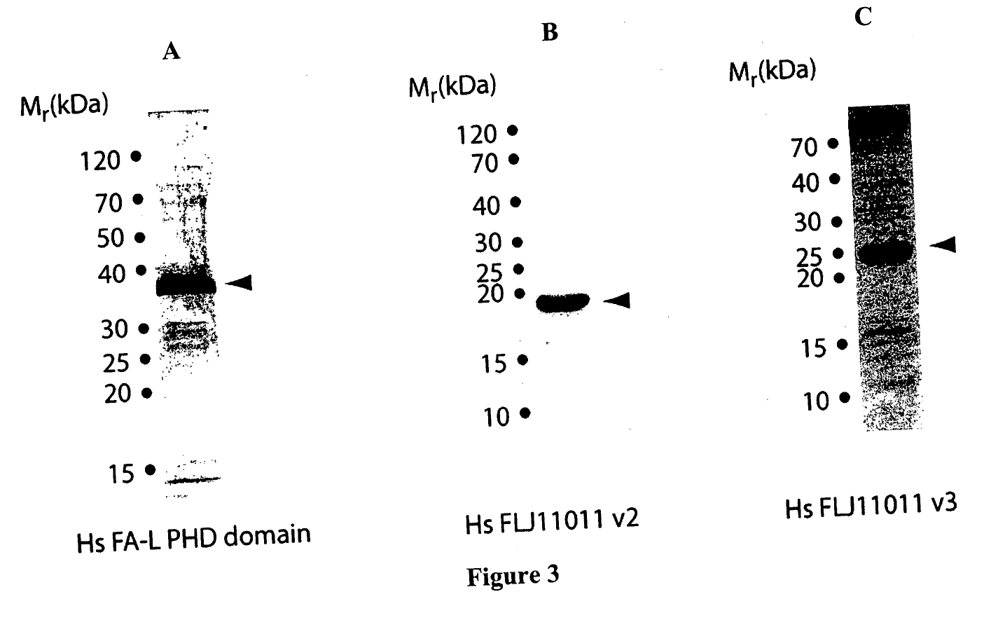

Sequence analysis revealed putative homologs of CG7220 in the genome of several metazoan species including, C. elegans, Mus musculus, Homo sapiens and Tetraodon nigriviridis. The human homolog was called FLJ11011 and further sequence analyses revealed that mRNA encoding the human homolog occurs as three variant forms. Detection of the mRNA by RT-PCR using total RNA from human foreskin fibroblasts identified three differently sized mRNA species (FIG. 3). cDNA encoding the three putative forms of FLJ11011 were isolated by RT-PCR and cloned for protein expression in E. coli. Two forms of FLJ11011 (v2 and v3) were purified and analyzed for purity by SDS-PAGE (FIG. 3, lanes 2 and 3). Additionally, cDNA encoding the PHD-domain of human FA-L was obtained (Weidong Wang NIH, Baltimore, Md.) and protein encoded by this CDNA was expressed in, and purified from, E. coli (FIG. 3, lane 1).

### Example 3

### One of the E2 Variants, FLJ11011-v2, can accept Ub from human E1

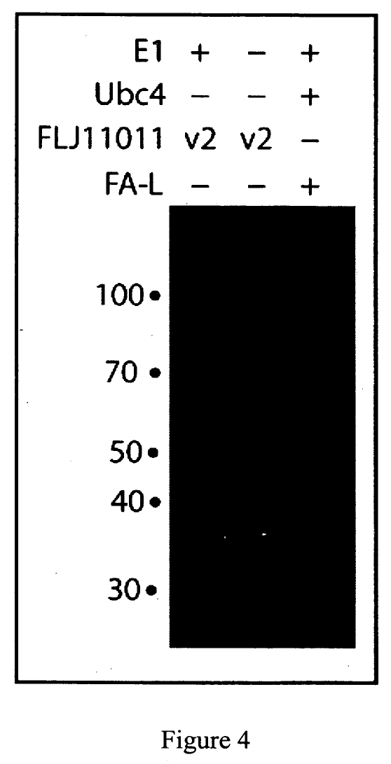

The purified proteins were used in ubiquitinylation reactions as in Example 2, however the reactions now contained Human E1, Human Ubc4 (E2) or variants of FLJ11011 and Human FA-L (E3). FLJ11011v2 is efficiently ubiquitinylated in this assay (FIG. 4, lane 1). This is a covalent modification as it survived boiling in SDS buffer that contains a reducing agent, but not dependent on an E3 (FA-L) enzyme. The ability to accept Ub from an Ub-activating enzyme (E1) in the presence of ATP is a criteria used to identify E2 enzymes.

The foregoing description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. An isolated nucleic acid molecule having a sequence selected from the group consisting of SEQ ID NO:1, SEQ ID NO:2, SEQ ID NO:3, and SEQ ID NO:4.

2. An isolated nucleic acid molecule which hybridizes under stringent conditions to a nucleic acid molecule selected from the group consisting of SEQ ID NO:1, SEQ ID NO:2, SEQ ID NO:3, and SEQ ID NO:4.

3. The nucleic acid molecule of claim 1, operably linked to a promoter element.

4. The nucleic acid molecule of claim 1, comprised in a vector.

5. The nucleic acid molecule of claim 4, wherein the nucleic acid molecule is operably linked to a promoter element.

6. A protein encoded by the nucleic acid molecule of claim 1.

7. A peptide fragment of the protein of claim 6 comprising at least 20 amino acids that cross-react with an immunoglobulin which specifically binds to a full-length FLJ11011 or CG7220 protein.

8. A protein having an amino acid sequence of SEQ ID NO:5.

9. An antibody which binds to a protein encoded by the nucleic acid molecule of claim 1.

10. A nucleic acid vector comprising an isolated nucleic acid having the nucleic acid sequence selected from the group consisting of SEQ ID NO:1, SEQ ID NO:2, and SEQ ID NO:3, operatively linked to a heterologous regulatory element and a polyadenylation signal.

11. A nucleic acid construct comprising a first nucleic acid sequence homologous to a portion of the FLJ11011 gene and a second nucleic acid sequence homologous to a second portion or region of the FLJ11011 gene and a positive selection marker positioned between the first and the second nucleic acid sequences.

12. The nucleic acid construct of claim 11, wherein the positive selection marker is operatively linked to a promoter and a polyadenylation signal.

13. A stem cell comprising a disruption in a gene selected from the group consisting of FLJ11011 and CG7720.

14. The stem cell of claim 13, wherein the stem cell is a murine stem cell.

15. A transgenic mammal comprising a disruption in a FLJ11011 gene wherein when the disruption is homozygous, the transgenic mammal lacks production of functional FLJ11011 protein.

16. A cell isolated from the transgenic mammal of claim 15.

17. The cell of claim 16, further comprising a nucleic acid construct comprising a recombinase gene operably linked to a functional promoter element.

18. An isolated cell comprising a FLJ11011 gene capable of expressing proteins and a detectable marker under the control of an inducible promoter.

19. A method of identifying an agent effective for the prevention, treatment or amelioration of symptoms of Fanconi's Anemia comprising:
administering an effective amount of a putative therapeutic agent to a transgenic animal comprising a disruption in a FLJ11011 gene;
comparing the response of the transgenic animal to a control animal having a functional FLJ11011 gene, wherein a response by the transgenic animal indicative of overcoming or lessening in the symptoms of Fanconi's anemia is indicative of effective treatment of Fanconi's anemia by the agent.

20. A method of identifying an agent effective for the prevention, treatment or amelioration of symptoms of cancer comprising:
administering an effective amount of a putative therapeutic agent to a transgenic animal comprising a disruption in a FLJ11011 gene;
comparing the response of the transgenic animal to a control animal having a functional FLJ11011 gene, wherein a response by the transgenic animal is indicative of effective treatment or prevention of cancer by the agent.

21. A method of identifying an agent effective for the prevention, treatment or amelioration of symptoms of Fanconi's Anemia comprising:
contacting an isolated cell comprising a FLJ11011 gene capable of expressing proteins and a detectable marker under the control of an inducible promoter with a putative therapeutic agent;
comparing a cellular phenotype of Fanconi's Anemia of the isolated cell with the phenotype of a control cell lacking a functional FLJ11011 gene, wherein a response by the isolated cell is indicative of an agent that ameliorates symptoms of Fanconi's Anemia.

22. A method of identifying an agent effective for the prevention, treatment or amelioration of symptoms of cancer comprising:
contacting an isolated cell comprising a FLJ11011 gene capable of expressing proteins and a detectable marker under the control of an inducible promoter with a putative therapeutic agent;
comparing a cellular cancer phenotype of the isolated cell with the phenotype of a control cell lacking a functional FLJ11011 gene, wherein a response by the isolated cell is indicative the prevention or treatment of cancer by the agent.

23. A method of producing an antibody to a ubiquitin-conjugating (E2) enzyme comprising:
injecting a compound selected from the group consisting of a FLJ11011 gene, a CG7220 gene, a FLJ11011 protein, a CG7220 protein, and fragments thereof into a host animal; and,
isolating from the host animal an antibody that specifically recognizes the compound.

24. A method of testing a mammal for increased susceptibility to cancer comprising:
obtaining a tissue sample from the mammal,
testing the tissue sample for the presence of a mutation in an FLJ11011 gene.

25. A method of testing a mammal for increased susceptibility to cancer comprising:
obtaining a tissue sample from the mammal,
testing the tissue sample for the presence FLJ11011 protein activity in a ubiquitin conjugating assay.

26. A method of diagnosing Fanconi's Anemia (FA) or a predisposition to develop cancer comprising:
detecting in a tissue sample from a patient to be tested the level of expression of FLJ11011,
comparing the level of expression of the FLJ11011 detected in the patient sample to a level of expression of FLJ11011 that has been associated with FA and a level of expression of FLJ11011 that has been associated with normal controls and,
diagnosing FA in the patient if the expression level of FLJ11011 in the patient sample is statistically more similar to the expression level of the FLJ11011 that has been associated with FA than the expression level of the FLJ11011 that has been associated with the normal controls.


SEQUENCE LISTING (5 sequences)

SEQ ID NO: 1 - DNA, 4057 bp, Homo sapiens
agctcctccccctgcgtctctggcctcgccggtcttggggggatggttccatcatggcgtcaatgcagaccacaggaaggagggtggaagtatggtttccaaaacgactacagaaagaactgttggctttgcaaaatgacccacctcctggaatgaccttaaatgagaagagtgttcaaaattcaattacacagtggattgtagacatggaaggtgcaccaggtaccttatatgaaggggaaaaatttcaacttctatttaaatttagtagtcgatatccttttgactctcctcaggtcatgtttactggtgaaaatattcctgttcatcctcatgtttatagcaatggtcatatctgtttatccattctaacagaagactggtccccagcgctctcagtccaatcagtttgtcttagcattattagcatgctttccagctgcaaggaaaagagacgaccaccggataattctttttatgtgcgaacatgtaacaagaatccaaagaaaacaaaatggtggtatcatgatgatacttgttgatgccactgttatcatcctcctagcagaagatagtcctactgagaaaatgagcactttgatcattcagtctttgaactttaacctttgactggaagtgacctataggcaatgaagactacttccttttactgcatttttactcgtgtgcattctgggcgcatgttgatcgctggttcagtccaggcaactgacatgcttttattagtcatacagtattaatgcaggtgtcaggaaatgtcaaatataattccattttttatttttatttttttaagcttttggaaaagctccaggtcctcatgtattgtgcaataacaatgacttccttggcggttttggtacgttcattgccggcaatgggcgttgtaacaggaaaagttttcattaactcctgccattcaatgattaatgcatgatagggcctatgaaatgaacttactggttatagtgggaatataaataaagtgagggatccaacattactttaaaagtcaccccaactgtttatatttggattctatgcactgtgatcctaaggttaacagcatgaattaacatgcgtctttaaaggactgtaatgaaagatcattgcatatttattgaattgtttatatctactgtcaagttgttttgacatggaagattttcaagtaacattggcagagaggtacagtatgttatccctatggtgaaaataaattaatttgttgtatatagttcctcaatctctgaagtaaaggtatgagtaatatagggtatgaatggtttaatcaaggctttattttggaagtaagaaaaatggcagtgatgattaaattgctgcagtccataatttgggcttgttatttgtacattaaagattttttccaagtaagttacactctgttaacttcctgctagccatcagcatgagccctactgcctaaacactatttcatttatttatgtttggaaaccccgtaaacatttttgtttgcaatcttgtttcttttgttataagtcaagtttgaatgttacaatacttttattgaaacttttgttaagttttttcttgtaaattttctttacttgtgagtatcatcttgtcctttaatcctgtaccctaaaataagaaatacatttttgacagaggcttaatgttttaacaaaagagtgtggacatttttattttaaaatttaggcaaaagtcactatcaaatggttgcttatttgtctcacacagccatatagtttttcctggagggttttgttttgttgttgttgaaaagactttgcttacagctagatgaaactttctatagaaaaaaaaaattgttgaaaggtccagttctcagtaccatgtgagttaatgatactacaactaagttctttttaaaaagtgattaatgtattttataaattaccttttcacatatgcaaaatctgtttctactacaatgttatttttactaatgccttattgttgcactctttttgaaatatcctgcagtgaatatatgaatcaatttgggcttaaaactgaaagccagttggctgaaaggtttgaaatacgtaccccagtaaaaccattcaatcaataattggtaaataatattttaaaattgtttttaatctgtatagatgacattttgtagctttgtacatgttgttaattaagggcatataattttacactcaaagtataattgctgaactcaggggtgggtagacttcaaaaatatgtctgctatagaaataacttgaaaaaaaaaattaaaaccgtggccagccaccaaattgtggacactgtttatatactgctagttaacaattaccacttttaaaaaattataaccgtttttgttctatcttgactagataaattctcacaatgatctctgtcagttttattagttggcctgtttaaccaagacagaaagtctttgaattactcatagttttaaactgcttttaatatgaaaattgcagctgtaaattatgtattttcatattttgcgtaactgctctacactactgtttgacttgatcttcttctgggggttagggagaaatctgtctccatattgctggttctcttaccaaaatgcttttataaagaaatgaccggtgacatttattcaccaaaggaattaatatactgagtcaaagattaacaatgctatactatagagtaataggtcatatatagcctcaattgagttttttatgacaatattttaacatacctctctctctacatataaataccataaagtaaactaaaatgacacagaggaaagtagaggaaaatgaaataatttcggtacatcttatggtttaaagagtagaaaataagatgaaatatcctaaaatataggagcacagacatttaaaattcataatcaggctaacaacactctcttgaatttaggaatcattgaaaactccttgcacttagatttaatttatatgttagttacattattaaaagtcataaggcatttatctgagtcattgctctacagagagcaattaatggcttgtaataataaaaagaatatttctctgtaatgcattgttgaaagacaaccagaatatcaagaaaatgtatataggaatatgctacttgcctcctccctgcatttcctcgtggggcactatttgcgacataggcctacagaattagactttcatacttagaagataaggtacactcttcttcttttatttatttatttatttatttttaggccattcttctggtttgccaaattctattaagacagtgatacatttgcttccttatttatgcatgcttagatcatgaacatgatgcccttcttttttttttttttctttggtcaagtagggattaatgccataggtttagagagaaggattgttttcagtttttaagagaattttttaaaaatacattttatggttctcttaaggaattaatattcacttctgaaggacttacttgtaatatataatttatcattttagccagcagggaaataaatcatttttaatccccagaagaaagtgctggaaatagcttctatatagtcagcagttcacctggagaaagatcttaattccttgttgttttgactaggaatatcaatatttggtataaaataactagaatagtttattaccttttccctcatgaaattttaaatatatgcaaatatatatatcattagattcctgtggtggataataatttcttcagattctaaaacaggtgtctagtgactgcttaattgtttccaagttgcatcaaagatgaattggtaaataatggaaatgagtagtgatatgaaaatggttatgtaactacatattacttgtttctggtaatcaagtcttattttttaaaagcctattgtttttcagaattgagatatttaaaacttgttataacttcttcctaaaaatagaagagaagaggaggtactgtgtgttacaaaaatttttttaaataaagaattcagtgtcactagttctaaaaaaaaaaaaaaaaaaca

SEQ ID NO: 2 - DNA, 4024 bp, Homo sapiens
agctcctccccctgcgtctctggcctcgccggtcttggggggatggttccatcatggcgtcaatgcagaaacgactacagaaagaactgttggctttgcaaaatgacccacctcctggaatgaccttaagtgagaagagtgttcaaaattcaattacacagtggattgtagacatggaaggtgcaccaggtaccttatatgaaggggaaaaatttcaacttctatttaaatttagtagtcgatatccttttgactctcctcaggtcatgtttactggtgaaaatattcctgttcatcctcatgtttatagcaatggtcatatctgtttatccattctaacagaagactggtccccagcgctctcagtccaatcagtttgtcttagcattattagcatgctttccagctgcaaggaaaagagacgaccaccggataattctttttatgtgcgaacatgtaacaagaatccaaagaaaacaaaatggtggtatcatgatgatacttgttgatgccactgttatcatcctcctagcagaagatagtcctactgagaaaatgagcactttgatcattcagtctttgaactttaacctttgactggaagtgacctataggcaatgaagactacttccttttactgcatttttactcgtgtgcattctgggcgcatgttgatcgctggttcagtccaggcaactgacatgcttttattagtcatacagtattaatgcaggtgtcaggaaatgtcaaatataattccattttttatttttatttttttaagcttttggaaaagctccaggtcctcatgtattgtgcaataacaatgacttccttggcggttttggtacgttcattgccggcaatgggcgttgtaacaggaaaagttttcattaactcctgccattcaatgattaatgcatgatagggcctatgaaatgaacttactggttatagtgggaatataaataaagtgagggatccaacattactttaaaagtcaccccaactgtttatatttggattctatgcactgtgatcctaaggttaacagcatgaattaacatgcgtctttaaaggactgtaatgaaagatcattgcatatttattgaattgtttatatctactgtcaagttgttttgacatggaagattttcaagtaacattggcagagaggtacagtatgttatccctatggtgaaaataaattaatttgttgtatatagttcctcaatctctgaagtaaaggtatgagtaatatagggtatgaatggtttaatcaaggctttattttggaagtaagaaaaatggcagtgatgattaaattgctgcagtccataatttgggcttgttatttgtacattaaagattttttccaagtaagttacactctgttaacttcctgctagccatcagcatgagccctactgcctaaacactatttcatttatttatgtttggaaaccccgtaaacatttttgtttgcaatcttgtttcttttgttataagtcaagtttgaatgttacaatacttttattgaaacttttgttaagttttttcttgtaaattttctttacttgtgagtatcatcttgtcctttaatcctgtaccctaaaataagaaatacatttttgacagaggcttaatgttttaacaaaagagtgtggacatttttattttaaaatttaggcaaaagtcactatcaaatggttgcttatttgtctcacacagccatatagtttttcctggagggttttgttttgttgttgttgaaaagactttgcttacagctagatgaaactttctatagaaaaaaaaaattgttgaaaggtccagttctcagtaccatgtgagttaatgatactacaactaagttctttttaaaaagtgattaatgtattttataaattaccttttcacatatgcaaaatctgtttctactacaatgttatttttactaatgccttattgttgcactctttttgaaatatcctgcagtgaatatatgaatcaatttgggcttaaaactgaaagccagttggctgaaaggtttgaaatacgtaccccagtaaaaccattcaatcaataattggtaaataatattttaaaattgtttttaatctgtatagatgacattttgtagctttgtacatgttgttaattaagggcatataattttacactcaaagtataattgctgaactcaggggtgggtagacttcaaaaatatgtctgctatagaaataacttgaaaaaaaaaattaaaaccgtggccagccaccaaattgtggacactgtttatatactgctagttaacaattaccacttttaaaaaattataaccgtttttgttctatcttgactagataaattctcacaatgatctctgtcagttttattagttggcctgtttaaccaagacagaaagtctttgaattactcatagttttaaactgcttttaatatgaaaattgcagctgtaaattatgtattttcatattttgcgtaactgctctacactactgtttgacttgatcttcttctgggggttagggagaaatctgtctccatattgctggttctcttaccaaaatgcttttataaagaaatgaccggtgacatttattcaccaaaggaattaatatactgagtcaaagattaacaatgctatactatagagtaataggtcatatatagcctcaattgagttttttatgacaatattttaacatacctctctctctacatataaataccataaagtaaactaaaatgacacagaggaaagtagaggaaaatgaaataatttcggtacatcttatggtttaaagagtagaaaataagatgaaatatcctaaaatataggagcacagacatttaaaattcataatcaggctaacaacactctcttgaatttaggaatcattgaaaactccttgcacttagatttaatttatatgttagttacattattaaaagtcataaggcatttatctgagtcattgctctacagagagcaattaatggcttgtaataataaaaagaatatttctctgtaatgcattgttgaaagacaaccagaatatcaagaaaatgtatataggaatatgctacttgcctcctccctgcatttcctcgtggggcactatttgcgacataggcctacagaattagactttcatacttagaagataaggtacactcttcttcttttatttatttatttatttatttttaggccattcttctggtttgccaaattctattaagacagtgatacatttgcttccttatttatgcatgcttagatcatgaacatgatgcccttcttttttttttttttctttggtcaagtagggattaatgccataggtttagagagaaggattgttttcagtttttaagagaattttttaaaaatacattttatggttctcttaaggaattaatattcacttctgaaggacttacttgtaatatataatttatcattttagccagcagggaaataaatcatttttaatccccagaagaaagtgctggaaatagcttctatatagtcagcagttcacctggagaaagatcttaattccttgttgttttgactaggaatatcaatatttggtataaaataactagaatagtttattaccttttccctcatgaaattttaaatatatgcaaatatatatatcattagattcctgtggtggataataatttcttcagattctaaaacaggtgtctagtgactgcttaattgtttccaagttgcatcaaagatgaattggtaaataatggaaatgagtagtgatatgaaaatggttatgtaactacatattacttgtttctggtaatcaagtcttattttttaaaagcctattgtttttcagaattgagatatttaaaacttgttataacttcttcctaaaaatagaagagaagaggaggtactgtgtgttacaaaaatttttttaaataaagaattcagtgtcactagttctaaaaaaaaaaaaaaaaaaca

SEQ ID NO: 3 - DNA, 3921 bp, Homo sapiens
agctcctccccctgcgtctctggcctcgccggtcttggggggatggttccatcatggcgtcaatgcagaaacgactacagaaagaactgttggctttgcaaaatgacccacctcctggaatgaccttaaatgagaagagtgttcaaaattcaattacacagtcatgtttactggtgaaaatattcctgttcatcctcatgtttatagcaatggtcatatctgtttatccattctaacagaagactggtccccagcgctctcagtccaatcagtttgtcttagcattattagcatgctttccagctgcaaggaaaagagacgaccaccggataattctttttatgtgcgaacatgtaacaagaatccaaagaaaacaaaatggtggtatcatgatgatacttgttgatgccactgttatcatcctcctagcagaagatagtcctactgagaaaatgagcactttgatcattcagtctttgaactttaacctttgactggaagtgacctataggcaatgaagactacttccttttactgcatttttactcgtgtgcattctgggcgcatgttgatcgctggttcagtccaggcaactgacatgcttttattagtcatacagtattaatgcaggtgtcaggaaatgtcaaatataattccattttttatttttatttttttaagcttttggaaaagctccaggtcctcatgtattgtgcaataacaatgacttccttggcggttttggtacgttcattgccggcaatgggcgttgtaacaggaaaagttttcattaactcctgccattcaatgattaatgcatgatagggcctatgaaatgaacttactggttatagtgggaatataaataaagtgagggatccaacattactttaaaagtcaccccaactgtttatatttggattctatgcactgtgatcctaaggttaacagcatgaattaacatgcgtctttaaaggactgtaatgaaagatcattgcatatttattgaattgtttatatctactgtcaagttgttttgacatggaagattttcaagtaacattggcagagaggtacagtatgttatccctatggtgaaaataaattaatttgttgtatatagttcctcaatctctgaagtaaaggtatgagtaatatagggtatgaatggtttaatcaaggctttattttggaagtaagaaaaatggcagtgatgattaaattgctgcagtccataatttgggcttgttatttgtacattaaagattttttccaagtaagttacactctgttaacttcctgctagccatcagcatgagccctactgcctaaacactatttcatttatttatgtttggaaaccccgtaaacatttttgtttgcaatcttgtttcttttgttataagtcaagtttgaatgttacaatacttttattgaaacttttgttaagttttttcttgtaaattttctttacttgtgagtatcatcttgtcctttaatcctgtaccctaaaataagaaatacatttttgacagaggcttaatgttttaacaaaagagtgtggacatttttattttaaaatttaggcaaaagtcactatcaaatggttgcttatttgtctcacacagccatatagtttttcctggagggttttgttttgttgttgttgaaaagactttgcttacagctagatgaaactttctatagaaaaaaaaaattgttgaaaggtccagttctcagtaccatgtgagttaatgatactacaactaagttctttttaaaaagtgattaatgtattttataaattaccttttcacatatgcaaaatctgtttctactacaatgttatttttactaatgccttattgttgcactctttttgaaatatcctgcagtgaatatatgaatcaatttgggcttaaaactgaaagccagttggctgaaaggtttgaaatacgtaccccagtaaaaccattcaatcaataattggtaaataatattttaaaattgtttttaatctgtatagatgacattttgtagctttgtacatgttgttaattaagggcatataattttacactcaaagtataattgctgaactcaggggtgggtagacttcaaaaatatgtctgctatagaaataacttgaaaaaaaaaattaaaaccgtggccagccaccaaattgtggacactgtttatatactgctagttaacaattaccacttttaaaaaattataaccgtttttgttctatcttgactagataaattctcacaatgatctctgtcagttttattagttggcctgtttaaccaagacagaaagtctttgaattactcatagttttaaactgcttttaatatgaaaattgcagctgtaaattatgtattttcatattttgcgtaactgctctacactactgtttgacttgatcttcttctgggggttagggagaaatctgtctccatattgctggttctcttaccaaaatgcttttataaagaaatgaccggtgacatttattcaccaaaggaattaatatactgagtcaaagattaacaatgctatactatagagtaataggtcatatatagcctcaattgagttttttatgacaatattttaacatacctctctctctacatataaataccataaagtaaactaaaatgacacagaggaaagtagaggaaaatgaaataatttcggtacatcttatggtttaaagagtagaaaataagatgaaatatcctaaaatataggagcacagacatttaaaattcataatcaggctaacaacactctcttgaatttaggaatcattgaaaactccttgcacttagatttaatttatatgttagttacattattaaaagtcataaggcatttatctgagtcattgctctacagagagcaattaatggcttgtaataataaaaagaatatttctctgtaatgcattgttgaaagacaaccagaatatcaagaaaatgtatataggaatatgctacttgcctcctccctgcatttcctcgtggggcactatttgcgacataggcctacagaattagactttcatacttagaagataaggtacactcttcttcttttatttatttatttatttatttttaggccattcttctggtttgccaaattctattaagacagtgatacatttgcttccttatttatgcatgcttagatcatgaacatgatgcccttcttttttttttttttctttggtcaagtagggattaatgccataggtttagagagaaggattgttttcagtttttaagagaattttttaaaaatacattttatggttctcttaaggaattaatattcacttctgaaggacttacttgtaatatataatttatcattttagccagcagggaaataaatcatttttaatccccagaagaaagtgctggaaatagcttctatatagtcagcagttcacctggagaaagatcttaattccttgttgttttgactaggaatatcaatatttggtataaaataactagaatagtttattaccttttccctcatgaaattttaaatatatgcaaatatatatatcattagattcctgtggtggataataatttcttcagattctaaaacaggtgtctagtgactgcttaattgtttccaagttgcatcaaagatgaattggtaaataatggaaatgagtagtgatatgaaaatggttatgtaactacatattacttgtttctggtaatcaagtcttattttttaaaagcctattgtttttcagaattgagatatttaaaacttgttataacttcttcctaaaaatagaagagaagaggaggtactgtgtgttacaaaaatttttttaaataaagaattcagtgtcactagttctaaaaaaaaaaaaaaaaaaca

SEQ ID NO: 4 - DNA, 1756 bp, Homo sapiens
ccattctagggaccacaacaacttttctaaactgtcgtcagtataacttgtcaacccaaattggattgacaaatttttaaaacaattattgtactccgctgaaattaaactatttaccggcgttcaaagcggaaaactcaaaaatacctacacgcggaacagctgagtgcgagctttggccacggaggaaacggaacagatagccagcgagcggagcagccaggacttggagcaggaatcggcaggaacaccaggacggatttccccaagacaagcagtagtagtatgtcttcgaagtcgccaagcgagcgccgcctacacaaagagctgatgtccctgatcaaggagccgccaccaggcgtaaccatcgacaccgagagcgtacaacaaaatttatccgaatggaaaataaacattaaaggtttcgagggcacactttacgagggcgaggacttccagctacttttcaaattcaacaataaatatccgttcgattcgccagaggtgaccttcatcggcaccaatataccggtgcatccgcatgtctacagcaacggacacatctgcctatccattctgaccgaggactggtccccggcgctgtccgtgcagtccgtgtgccttagcatagcctccatgttgagtagttgtcgcgaaaagaagcgtccgccggacaacacgatatacgtaaaaacctgtaataagaatcctaaaaagactaaatggtggtaccacgatgattctgtttagttaaaattgccatttactaatactgcctgctattgtttgctttgcgcatccaaccaaaaaaaaaaaaaaaaaaaaaaacaccattagcaatacagaagtagaacacggcgagatgcccccagaaatccaagaaatgtgggacggcggtcaagcactgctcgtgagtggagcatactaaaacccaagtgcactcggatgcgaacgtaatgtgcaacaaagtaaatgaagttagcgagaaacttatgggtagtgtttggcaaacaatcgtggcgcatgcaaacaattctaattgtagctatgtgtaatcaacaaaaggcgtgtgcgaaaaactattattaaatgttaacattttaattagattgttaagaccaaatttatgtctaaattaaatgttatcctaccatttaaccactaagcgtatgcaaaccacttgaacgtttgatagttctggcacctcgcgccagttgctgcttccacattccacatgcatcgttcgcggcattttctgcccgagtagcgtgacccagcacacacacacatcgcctccatcccagcttatattaattttgtgtattttatcatcatagatgagaagcaaaactcatgcgatcatagttaagattatgacattttaaatgtattagcagccatatccccgatcgctctcattgtcaccagtcctgcactctgtaatctacccacattgcccatgcataggtcgttaagttaaccagttcatgtacttggctaggatagtccatctgtggccaaaaatctcgcaagtggcccgatcgcagaacacatttacctctaactgtgcaagtagatcttaggatgtgacgagctactcgtccgactatcacacgatttaactataatccgtataatgtataaagagcaagttaaatccaatgtatgtttatccagcatatgtctataaatgcctataaatatttactgttaaaagtatttaaaataaaaagaagtctgtagtcgaacgaaccaa

SEQ ID NO: 5 - protein, 151 aa, Homo sapiens
MASMQKRLQKELLALQNDPPPGMTLNEKSVQNSITQWIVDMEGASGTLYEGEKFQLLFKFSSRYPFDSPQVMFTGENIPVHPHVYSNGHICLSILTEDWSPALSVQSVCLSIISMLSSCKEKRRPPDNSFYVRTCNKNPKKTKWWYHDDTC